(19) United States
(12) Patent Application Publication
(10) Pub. No.: US 2025/0032511 A1
(43) Pub. Date: Jan. 30, 2025

(54) GANAXOLONE FOR USE IN TREATMENT OF SUPER REFRACTORY STATUS EPILEPTICUS

(71) Applicant: MARINUS PHARMACEUTICALS, INC., Radnor, PA (US)

(72) Inventor: Henrikas VAITKEVICIUS, Boston, MA (US)

(21) Appl. No.: 18/785,310

(22) Filed: Jul. 26, 2024

Related U.S. Application Data
(60) Provisional application No. 63/516,212, filed on Jul. 28, 2023.

(51) Int. Cl. A61K 31/57; A61K 9/00; A61P 25/08

(52) U.S. Cl. CPC A61K 31/57; A61K 9/0019; A61P 25/08

(57) ABSTRACT

This invention relates to methods for treating super refractory status epilepticus by administering ganaxolone.

### BACKGROUND

Status epilepticus (“SE”) is a life-threatening neurological emergency associated with significant morbidity and mortality (Betjemann and Lowenstein, (2015) The Lancet Neurology, 14 (6): 615-624). SE is manifested by prolonged seizure activity, typically persisting more than 5 minutes, or recurrent seizures without recovery of consciousness between seizures. Id. SE requires aggressive treatment to stop the seizure and prevent neurological damage, including neuronal death. SE becomes more difficult to control as its duration increases, and prolonged SE and refractoriness to treatment are associated with poor prognosis (Cherian & Thomas (2009), Ann. Indian. Acad. Neurol., 12 (3): 140-153). Goals of treatment include rapid seizure cessation, maintenance of seizure control, preventing progression to anesthetics and avoiding further medical complications. Id.

Super-refectory SE (“SRSE”) is the most severe form of SE and is associated with long-term neurological dysfunction. SRSE occurs in 23% to 43% of patients with SE and is associated with a high mortality of up to 50% (Lionel and Hrishi, Indian J. Crit. Care Med. 20(10): 587-592 (2016)). SRSE is SE that continues or recurs despite therapy with general anesthesia using anesthetic antiepileptic drugs, such as midazolam, propofol, ketamine, or barbiturate, for more than 24 hours or recurrence of SE on weaning of general anesthetics. Shorvon S, Ferlisi M. Brain 2011:134-2802-2818. These patients are severely ill and mortality is high.

Under current standards of care, most SE patients are first treated with first-line therapeutic agents, typically with benzodiazepines (e.g., diazepam, lorazepam, and midazolam) (Trinka and Kälviäinen (2017), Seizure, 44:65-73: Glauser et al., (2016), Epilepsy Curr. 26 (1): 48-61). If the patient does not respond, the patient is then treated with other, second-line, anti-seizure drugs. Id. If the seizure persists after one second-line drug, the patient is diagnosed as having refractory SE. After failure of second-line therapy, the patient is admitted to the ICU and placed into a medically induced coma. Id. Physicians typically use one or more anesthetic agents (e.g., thiopental, pentabarbital, propofol, and midazolam) to induce the coma, along with antiepileptic drugs in an attempt to stop the ongoing seizure in such refractory SE patients. Id. After a period of 24 hours, an attempt is usually made to slowly wean the patient from the anesthetic agents to evaluate whether or not the seizure condition has resolved. Unfortunately, not all patients respond to weaning attempts, and in those who do not seizure returns, and such patients are considered to have SRSE and must be maintained in the medically induced coma. In many instances, these patients die without further intervention. It is estimated that there are between 25,000 and 41,000 cases of SRSE in the U.S. each year.

Currently, there are no therapies specifically approved for SRSE. Typically SRSE patients are treated with anesthetics (frequently a two or more anesthetics) that are administered to place and maintain the SRSE patient in burst suppression with no ictal activity. Unfortunately, the brain adjusts to this type of treatment over a period of time and when anesthetics are reduced or withdrawn, the patient frequently shows hyperactive brain activity including recurrence of seizure.

WO 2022/109440 discloses the administration of ganaxolone as a therapeutic bridge in the treatment of SRSE. In that approach, SRSE patients are rapidly weaned off anesthetic agents and ictal activity is briefly allowed to return, then ganaxolone is administered to achieve a ganaxolone plasma concentration of about 800 ng/ml to about 1200 ng/ml to manage ictal activity while anesthetic agents continue to be withdrawn.

There is a need for effective therapies for treating SRSE.

### SUMMARY

The disclosure relates to a new paradigm and methods of treating SRSE. As described herein, the inventors have discovered that administering ganaxolone at lower doses while continuing to administer intravenous anesthesia to the patient, as opposed to rapidly weaning intravenous anesthesia and administering higher doses of ganaxolone, can provide effective therapy for SRSE. Without being bound by any particular theory, the inventors believe that starting administration of ganaxolone to SRSE patient while the patient is receiving anesthetic agents allows less ganaxolone to be initially administered followed by a steady state infusion to achieve a target serum ganaxolone concentration of at least about 500 ng/ml throughout the treatment period which enables the patient to be weaned off intravenous anesthesia without seizure recurrence. This approach provides many advantages. Notably the new treatment paradigm and methods are implemented using a relatively simple dosage regimen and steady state continuous infusion during the treatment period, and provides improved clinical outcomes in comparison to other treatment regimens.

In some practices of the method of this disclosure intravenous ganaxolone is administered to a subject in need thereof as an intravenous bolus plus continuous infusion. The subject in need thereof, typically has status epilepticus and is receiving intravenous anesthesia. When the subject in need thereof weighs 40 kg or more the intravenous bolus comprises about 15 mg to about 25 mg ganaxolone, and the continuous infusion comprises an infusion of about 50 mg to about 70 mg ganaxolone/hr for about 1 hour and then the ganaxolone dose is reduced to about 38 mg to about 52 mg ganaxolone/hr for about 23 hours. In preferred aspects, an intravenous bolus of 20 mg is administered over about 3 minutes, a continuous infusion of about 60 mg/hr is administered for about 1 hour (i.e., about 60 minutes) and then the infused dose is reduced to about 42 mg/hr for the remainder of the first day of treatment (i.e., about 23 hours).

When the subject in need thereof weighs less than 40 kg the bolus comprises about 0.25 to about 0.75 mg ganaxolone/kg, and the continuous infusion comprises an infusion of about 1.2 mg to about 2.0 mg ganaxolone/kg/hr for about 1 hour and then the ganaxolone dose is reduced to about 0.9 mg to about 1.2 mg ganavolone/kg/hr for about 23 hours. In preferred aspects, an intravenous bolus of 0.5 mg/kg is administered over about 3 minutes, a continuous infusion of about 1.5 mg/kg/hr is administered for about 1 hour (i.e., about 60 minutes) and then the infused dose is reduced to about 1.05 mg/kg/hr for the remainder of the first day of treatment (i.e., about 23 hours). For each group of subjects, those who weight weighs 40 kg or more and those who weigh less than 40 kg, the continuous infusion is then continued for an additional 24 to about 96 hours.

Beginning at about 4 hours after the start of the continuous infusion, intravenous anesthesia is weaned, and after about 48 hours to about 120 hours the intravenous anesthesia is discontinued. When intravenous anesthesia is discontinued, the intravenous ganaxolone is tapered over a period of about 2 days to about 4 days. The taper period is typically adjusted so that intravenous ganaxolone is discontinued no later than about 7 days after the initiation of intravenous ganaxolone therapy with the administration of the intravenous bolus on day 1 of therapy. The taper is performed by decreasing the amount of ganavolone that is infused every 4 hours in even increments to achieve discontinuation. The taper is complete and the continuous infusion is discontinued when the amount of ganaxolone that is infused is reduced to about 10 mg/hr. In embodiments, the taper begins on day 3, 4 or 5 after the intravenous bolus was administered and the taper is complete and the continuous infusion is discontinued on day 7 after the intravenous bolus was administered.

In some aspects, the method further comprises administering oral ganaxolone to the subject in need thereof after intravenous ganaxolone has been discontinued. When oral ganaxolone is desired or indicated, it is recommended that the subject start oral ganavolone (via a NG tube if needed) during intravenous ganaxolone taper. Oral ganaxolone is administered in a therapeutically effective amount, and typically at a dose of 1,800 mg/day for subjects that weigh more than 28 kg; and at a dose of 63 mg/kg for subjects that weigh 28 kg or less. In embodiments, oral ganaxolone is administered 3 divided doses each day. Oral ganaxolone can be administered for about 3 to about 6 months. In embodiments, oral ganaxolone is administered for about 3-6 months to subjects that weigh more than 28 kg, or for about 3 months to subjects that weigh 28 kg or less. When oral ganaxolone is no longer desired or indicated, it is tapered over a period of 2-3 weeks and discontinued.

### BRIEF DESCRIPTION OF DRAWINGS

FIG. 1 is a graph showing simulated ganaxolone (GNX) serum concentrations in a subject treated with intravenous GNX in accordance with embodiments of this disclosure in which intravenous GNX is administered as a 20 mg bolus and 60 mg/hr infusion for 60 minutes followed by 42 mg/hr for the remainder of the 24 hour simulation period. The regimen produces a steady state serum concentration of GNX of at least 500 ng/ml after about 2 to about 6 hours that is maintained through the treatment period.

FIG. 2 is a graph showing predicted and observed plasma ganaxolone concentrations.

FIGS. 3A-3F show EEG data. FIG. 3A is a bipolar montage EEG prior to GNX infusion; FIG. 3B is a quantitative EEG and the time-mark indicated that start of GNX infusion; FIG. 3C is a bipolar montage EEG at one hour post-GNX infusion; FIG. 3D is a bipolar montage EEG at twelve hours post-GNX infusion; FIG. 3E is a bipolar montage EEG at 5 days post-GNX infusion; FIG. 3F is a quantitative EEG at five days post-GNX infusion.

### DETAILED DESCRIPTION

The disclosure relates to a new paradigm and methods of treating SRSE. As described herein, the inventors have discovered that administering ganaxolone at lower doses while continuing to administer intravenous anesthesia to the patient, as opposed to rapidly weaning intravenous anesthesia and administering higher doses of ganaxolone, can provide effective therapy for SRSE. Without being bound by any particular theory, it is believed that starting administration of ganaxolone to SRSE patient while the patient is receiving anesthetic agents allows less ganaxolone to be initially administered followed by a steady state to achieve a predicted target serum ganaxolone concentration of at least about 500 ng/ml throughout the treatment period which enables the patient to be weaned off intravenous anesthesia without seizure recurrence. Ganaxolone has been used to treat SRSE using other therapeutic regimens. In one such regimen, intravenous anesthesia was weaned until seizures started and then intravenous ganaxolone was administered in higher doses than the in the method disclosed herein (See, WO 2022/109440). That approach showed early success, but seizures returned in some patients when ganaxolone serum levels were reduced, which occurred as early as day 1 of treatment. Based on these results and without wishing to be bound by theory, it is believed that treatment with concurrent intravenous anesthesia and intravenous ganaxolone, which occurs over a period of about 2 to about 5 days when intravenous ganaxolone is started and continuing while intravenous anesthesia is slowly weaned according to the methods described herein, is synergistic and provides improved clinical outcomes. This approach provides many advantages. Notably the new treatment paradigm and methods are implemented using a relatively simple dosage regimen and steady state continuous infusion during the treatment period, and provides improved clinical outcomes in comparison to other treatment regimens.

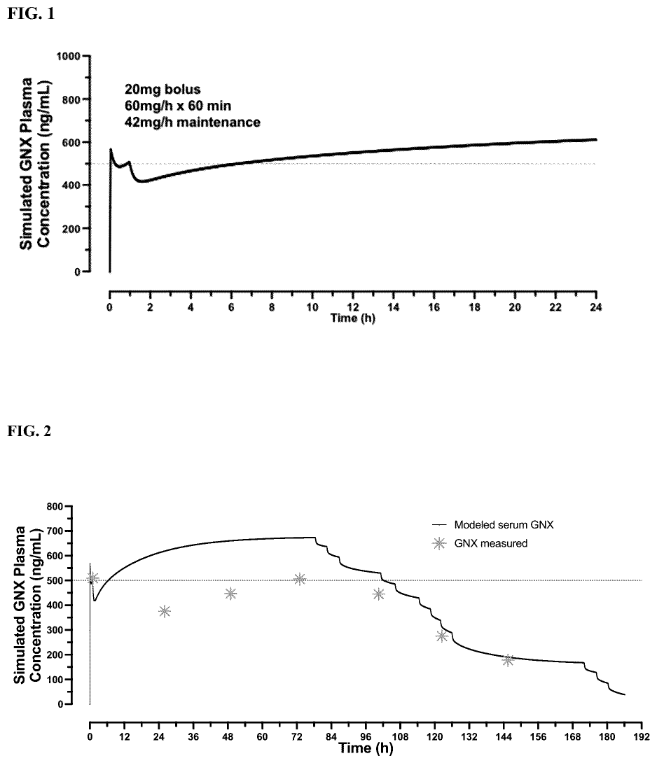

The methods described herein comprise administering intravenous ganaxolone to a subject with SRSE who is also receiving intravenous anesthesia, and typically one or more other anti-epileptic medications. The intravenous ganavolone is administered as a bolus and continuous infusion. Using this approach, a steady state serum concentration of ganavolone of at least about 500 ng/ml is achieved over a period of about 2 to about 6 hours and maintained through the treatment period. See, FIG. 1. The intravenous anesthetic is weaned starting at about 4 hours after initiation of the ganaxolone infusion and can continue until intravenous anesthetic is discontinued up to five days later, but preferably with 48 hours of beginning the wean. Upon discontinuation of the intravenous anesthetic, the intravenous ganaxolone is tapered and discontinued so that the total period of intravenous ganaxolone administration is no more than 7 days. If desired, the subject can be treated with oral ganaxolone after intravenous ganaxolone is discontinued. In such situations, oral ganaxolone is preferably started at the beginning of the intravenous ganaxolone taper. Typically subject weighing more that 28 kg are administered oral ganaxolone at a dose or 1,800 mg/day in three divided doses, and subject weighing 28 kg or less are administered oral ganaxolone at a dose of 63 mg/kg/day in three divided doses, but these doses can vary based on the judgement of the attending clinician.

Additional description of the method and guidance for the practice of the method are provided herein. For ease of presentation, further details and guidance are provided with respect to a preferred aspect using ganaxolone.

### Intravenous Ganaxolone Administration

In practice of the methods disclosed herein, intravenous ganaxolone is administered to a subject in need thereof. The subject in need thereof has super refractory status epilepticus and is receiving intravenous anesthesia, and typically also being treated with one or more other anti-epileptic agents. The intravenous ganaxolone is administered as a bolus and continuous infusion. The intravenous ganaxolone dosing according to the methods disclosed herein is different for subjects who weigh 40 kg or more and those who weigh less than 40 kg. Additionally, those who weigh 40 kg or more receive flat dosing while those who weigh less than 40 kg receive weight-based dosing. The period of time in which the subject receives intravenous ganaxolone is preferably no more than about 7 days. Accordingly, in preferred practices of the methods described herein, intravenous ganaxolone is discontinued after 7 days. In some instances, intravenous ganaxolone is discontinued after 6 days, or after 5 days, or after 4 days.

### Subjects Weighing 40 kg or More

Intravenous ganaxolone is started on day 1 with a bolus of about 15 mg to about 25 mg of ganaxolone. In examples, a bolus of about 15 mg, about 16 mg, about 17 mg, about 18 mg, about 19 mg, about 20 mg, about 21 mg, about 22 mg, about 23 mg, about 24 mg, or about 25 mg, is administered. Preferably, a bolus of about 20 mg of ganaxolone is administered. The intravenous bolus can be administered to the subject for any desired period of time and is typically administered from about 1 minute to about 10 minutes, such as, from about 1 minute to about 5 minutes, about 1 minute to about 4 minutes, about 1 minute to about 3 minutes, about 1 minute to about 2 minutes, about 2 minutes to about 5 minutes, about 2 minutes to about 4 minutes, about 2 minutes to about 3 minutes, about 3 minutes to about 5 minutes, or about 3 minutes to about 4 minutes. Preferably, the intravenous bolus is administered to the subject over a period of about 3 minutes.

On day 1, following the administration of the bolus a continuous infusion of ganaxolone is administered. The continuous intravenous infusion of ganaxolone can be administered periprocedural with the intravenous bolus. For instance, the continuous intravenous infusion can be initiated concurrently with the administration of the intravenous bolus. Alternatively, the continuous intravenous infusion can be initiated before or after the administration of the intravenous bolus. Typically, the intravenous bolus and continuous intravenous infusion is administered from the same ganaxolone source (e.g., and intravenous bag connected to an IV line).

The initial dose of the continuous infusion is about 50 mg/hr to about 70 mg/hr, and this initial dose is infused for about 1 hour. For example, the initial dose can be about 50 mg/hr, about 60 mg/hr, about 65 mg/hr, or about 70 mg/hr. Preferably, the continuous infusion begins with an initial ganaxolone dose of about 60 mg/h and is infused for about 1 hour. The ganaxolone dose administered in the continuous infusion is then reduced to a steady state or maintenance dose of about 38 mg/hr to about 52 mg/hr for the remainder of day 1 (i.e., about the next 23 hours). For example, the steady state or maintenance dose can be about 38 mg/hr, about 39 mg/hr, about 40 mg/hr, about 41 mg/hr, about 42 mg/hr, about 43 mg/hr, about 44 mg/hr, about 45 mg/hr, about 46 mg/hr, about 47 mg/hr, about 48 mg/hr, about 49 mg/hr, about 50 mg/hr, about 51 mg/hr, or about 52 mg/hr. Preferably, the steady state or maintenance dose of ganaxolone is about 42 mg/h and is infused for the remainder of day 1 (i.e., about the next 23 hours).

The continuous infusion of the steady state or maintenance dose of ganaxolone, which is preferably about 42 mg/h, is continued for the next 24-96 hours (treatment days 2-5) until the start of the ganaxolone taper.

Subjects Weighing Less than 40 kg

Intravenous ganaxolone is started on day 1 with a bolus of about 0.25 mg/kg to about 0.75 mg/kg of ganaxolone. In examples, a bolus of about 0.25 mg/kg, about 0.26 mg/kg, about 0.27 mg/kg, about 0.28 mg/kg, about 0.29 mg/kg, about 0.30 mg/kg, about 0.31 mg/kg, about 0.32 mg/kg, about 0.33 mg/kg, about 0.34 mg/kg, about 0.35 mg/kg, about 0.36 mg/kg, about 0.37 mg/kg, about 0.38 mg/kg, about 0.39 mg/kg, about 0.40 mg/kg, about 0.41 mg/kg, about 0.42 mg/kg, about 0.43 mg/kg, about 0.44 mg/kg, about 0.45 mg/kg, about 0.46 mg/kg, about 0.47 mg/kg, about 0.48 mg/kg, about 0.49 mg/kg, about 0.5 mg/kg, about 0.51 mg/kg, about 0.52 mg/kg, about 0.53 mg/kg, about 0.54 mg/kg, about 0.55 mg/kg, about 0.56 mg/kg, about 0.57 mg/kg, about 0.58 mg/kg, about 0.59 mg/kg, about 0.60 mg/kg, about 0.61 mg/kg, about 0.62 mg/kg, about 0.63 mg/kg, about 0.64 mg/kg, about 0.65 mg/kg, about 0.66 mg/kg, about 0.67 mg/kg, about 0.68 mg/kg, about 0.69 mg/kg, about 0.7 mg/kg, about 0.71 mg/kg, about 0.72 mg/kg, about 0.73 mg/kg, about 0.74 mg/kg, or about 0.75 mg/kg is administered. Preferably, a bolus of about 0.5 mg/kg of ganaxolone is administered. The intravenous bolus can be administered to the subject for any desired period of time and is typically administered from about 1 minute to about 10 minutes, such as, from about 1 minute to about 5 minutes, about 1 minute to about 4 minutes, about 1 minute to about 3 minutes, about 1 minute to about 2 minutes, about 2 minutes to about 5 minutes, about 2 minutes to about 4 minutes, about 2 minutes to about 3 minutes, about 3 minutes to about 5 minutes, or about 3 minutes to about 4 minutes. Preferably, the intravenous bolus is administered to the subject over a period of about 3 minutes.

On day 1, following the administration of the bolus a continuous infusion of ganaxolone is administered. The continuous intravenous infusion of ganaxolone can be administered periprocedural with the intravenous bolus. For instance, the continuous intravenous infusion can be initiated concurrently with the administration of the intravenous bolus. Alternatively, the continuous intravenous infusion can be initiated before or after the administration of the intravenous bolus. Typically, the intravenous bolus and continuous intravenous infusion is administered from the same ganaxolone source (e.g., and intravenous bag connected to an IV line).

The initial dose of the continuous infusion is about 1.2 mg/kg/hr to about 2.0 mg/kg/hr, and this initial dose is infused for about 1 hour. For example, the initial dose can be about 1.2 mg/kg/hr, 1.3 mg/kg/hr, 1.4 mg/kg/hr, 1.5 mg/kg/hr, 1.6 mg/kg/hr, 1.7 mg/kg/hr, 1.8 mg/kg/hr, 1.9 mg/kg/hr, or 2.0 mg/kg/hr. Preferably, the continuous infusion begins with an initial ganaxolone dose of about 1.5 mg/kg/h and is infused for about 1 hour. The ganaxolone dose administered in the continuous infusion is then reduced to a steady state or maintenance dose of about 0.9 mg/kg/hr to about 1.2 mg/kg/hr for the remainder of day 1 (i.e., about the next 23 hours). For example, the steady state or maintenance dose can be about 0.9 mg/kg/hr, 0.91 mg/kg/hr, 0.92 mg/kg/hr, 0.93 mg/kg/hr, 0.94 mg/kg/hr, 0.95 mg/kg/hr, 0.96 mg/kg/hr, 0.97 mg/kg/hr, 0.98 mg/kg/hr, 0.99 mg/kg/hr, 1.00 mg/kg/hr, 1.01 mg/kg/hr, 1.02 mg/kg/hr, 1.03 mg/kg/hr, 1.04 mg/kg/hr, 1.05 mg/kg/hr, 1.06 mg/kg/hr, 1.07 mg/kg/hr, 1.08 mg/kg/hr, 1.09 mg/kg/hr, 1.10 mg/kg/hr, 1.11 mg/kg/hr, 1.12 mg/kg/hr, 1.13 mg/kg/hr, 1.14 mg/kg/hr, 1.15 mg/kg/hr, 1.16 mg/kg/hr, 1.17 mg/kg/hr, 1.18 mg/kg/hr, 1.19 mg/kg/hr, or 1.2 mg/kg/hr. Preferably, the steady state or maintenance dose of ganaxolone is about 1.05 mg/kg/h and is infused for the remainder of day 1 (i.e., about the next 23 hours).

The continuous infusion of the steady state or maintenance dose of ganaxolone, which is preferably about 1.05 mg/kg/h, is continued for the next 24-96 hours (treatment days 2-5) until the start of the ganaxolone taper.

### Weaning and Discontinuing Intravenous Anesthesia

Subjects treated according to the methods disclosed herein are weaned off intravenous anesthesia beginning on day 1, and typically about 3 to about 10 hours after the intravenous ganaxolone continuous infusion is started. For example, the intravenous anesthesia wean can start about 3 hours, about 4 hours, about 5 hours, about 6 hours, about 7 hours, about 8 hours about 9 hours, about 10 hours or a longer period of time after the start of the ganaxolone continuous infusion. Preferably, the intravenous anesthesia wean begins about 4 hours after the start of the ganaxolone continuous infusion.

Once the intravenous anesthesia wean is started, the amount of intravenous anesthesia administered to the subject is reduced periodically according to the clinical judgement of the attending clinician and eventually the intravenous anesthesia is terminated. Typically the intravenous anesthesia is weaned and discontinued over a period of about 48 hours to about 120 hours (i.e., 2 days to about 5 days). In examples, intravenous anesthesia is weaned and discontinued over a period of a about 48 hours (i.e, about 2 days), about 72 hours (i.e., about 3 days), about 96 hours (i.e., about 4 days), or about 120 hours (i.e., about 5 days). When the wean and discontinuation of intravenous is completed, the intravenous ganaxolone is tapered and discontinued.

### Tapering and Discontinuing Intravenous Ganaxolone

For both groups of subjects, those who weigh 40 kg or more and those who weigh less than 40 kg, intravenous ganaxolone is tapered and then discontinued after intravenous anesthesia has been discontinued. The taper occurs over a period of about 2 days to about 4 days, and intravenous ganaxolone is discontinued no later than about 7 days after the initiation of therapy with the administration of the intravenous bolus on day 1. Intravenous ganaxolone is tapered by decreasing the amount of ganaxolone that is infused into the subject (e.g., reducing the infusion rate) every 4 hours in even increments until the infusion is discontinued. The taper is complete and the continuous infusion is discontinued when the amount of ganaxolone that is infused is reduced to about 10 mg/hr. In certain practices of the method, intravenous anesthesia is complete and the ganaxolone taper begins on day 3, day 4 or day 5 of the treatment, with the intravenous ganaxolone bolus being administered in day 1, and intravenous ganaxolone is discontinued on day 7 of treatment.

### Oral Ganaxolone Therapy

If desired or clinically indicated, oral ganaxolone can be administered to the subject following discontinuation of intravenous ganaxolone. In such cases, preferably oral ganaxolone is started at the beginning of the taper of intravenous ganaxolone. A therapeutically acceptable amount of ganaxolone is administered orally. The target dose for oral ganaxolone is 1,800 mg/day in subjects who weigh more than 28 kg, and is 63 mg/kg/day in subjects who weigh 28 kg or less. Dose adjustments may be indicated or desired, for example to meat the needs of particular subjects, and can be determined by a clinician of ordinary skill. However, the total daily does preferably does not exceed 1,800 mg/day in subjects who weigh more than 28 kg, or 63 mg/kg/day in subjects who weigh 28 kg or less. Oral ganaxolone can be administered in a single daily dose or in two, three or more divided daily doses. In some preferred embodiments, oral ganaxolone is administered in three evenly divided doses, for example 600 mg three times a day or 21 mg/kg three times a day. Oral ganaxolone therapy can continue for a suitable period as determined by the attending clinician. Typically, oral ganaxolone is administered to subjects who weigh more than 28 kg for about 3-6 months, and to subjects who weigh 28 kg or less for about 3 months. Following the oral ganaxolone treatment period in each subject group, oral ganaxolone is tapered over a period of 2-3 weeks and then discontinued. Recommended dosages for taper are presented in Tables 1 and 2.

TABLE 1
Oral dosing for subjects weighing more than 28 kg

| Per Dose (mg) | Daily (mg) |
|---|---|
| 600 | 1800 |
| 400 | 1200 |
| 200 | 600 |
| 100 | 300 |
| 50 | 150 |

TABLE 2
Oral dosing for subjects weighing 28 kg or less

| Per Dose (mg/kg) | Daily (mg/kg) |
|---|---|
| 21 | 63 |
| 14 | 42 |
| 8 | 24 |
| 4 | 12 |
| 2 | 6 |

### Ganaxolone and Formulations

Ganaxolone (alternatively known as 3α-hydroxy-3β-methyl-5α-pregnan-20-one, SPT3162, MD 9150000, CCD-1042, Mepalon, and 1042) is the subject of Investigational New Drug Application (IND) No. 129,433. The molecular formula of ganaxolone is C₂₂H₃₆O₂, and the chemical structure is:

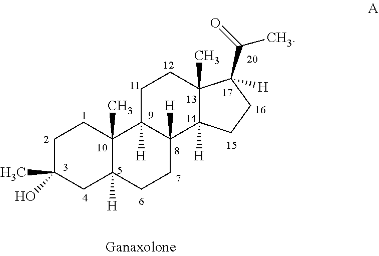

Ganaxolone (3α-hydroxy-3β-methyl-5α-pregnan-20-one) is a 3β-methylated synthetic analogue of the endogenous neurosteroid allopregnanolone with similar biological activity (Carter et al., (1997), The Journal of Pharmacology and Experimental Therapeutics, 280:1284-1295), but it is designed to not activate nuclear (classical) progesterone receptors. Also, in contrast to allopregnanolone, ganaxolone is orally bioavailable.

Any suitable formulations of ganaxolone can be used for intravenous or oral administration in accordance with the method described herein. Suitable intravenous and oral formulations are known in the art. See, e.g., U.S. Patent Publication No. 2017/0258812, U.S. Patent Publication No. 2016/0228454, U.S. Pat. Nos. 8,318,714, and 8,022,054.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “anesthesia” as used herein refers to the absence of all sensation. For example anesthesia results in amnesia, analgesia, muscle paralysis, and sedation.

The abbreviation “EEG” means electroencephalography.

The terms “subject” and “patient” are used interchangeably herein to refer to any animal, such as any mamma, including but not limited to, humans, non-human primates, rodents, and the like. In some embodiments, the mammal is a mouse. In some embodiments, the mammal is a human.

The term “effective amount” or “therapeutically effective amount” as used herein refers to an amount of a compound described herein (e.g., ganaxolone) that is sufficient to affect the intended result, including, but not limited to disease treatment as illustrated below. The “therapeutically effective amount” can be an amount effective to manage ictal activity (e.g., seizure activity), suppress SRSE, allow the patient to recover from a hyperexcitable state, prevents SE-relapse or can provide continued suppression of SRSE. The therapeutically effective amount can vary depending upon the intended application, or the subject and the disease condition being treated, e.g., the weight and age of the subject, the severity of the disease condition, the manner of administration and the like.

The term “pharmaceutical compositions” as used herein are compositions comprising at least one active agent, such as a compound or salt, solvate, or hydrate of ganaxolone, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as status epilepticus.

As used herein, the terms “treat,” treatment,” or “treating” and grammatically related terms, refer to an improvement of any sign, symptoms, or consequence of the disease, such as prolonged survival, less morbidity, and/or a lessening of side effects. As is readily appre

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### EXAMPLES

### Example 1: Therapeutic Regimen

The following therapeutic regimens are performed on patients with SRSE who are receiving intravenous anesthesia. Some patients will have failed at least two prior attempts to wean the intravenous anesthesia, and typically one of the failed attempts is within 24 hours of initiating the therapeutic regimen. The therapeutic regimen includes administration of intravenous ganaxolone for up to 7 days inclusive of taper. Intravenous anesthetic is weaned prior to the ganaxolone taper.

### Subjects Weighing 40 kg or More

On day 1 of the regimen, Ganaxolone is administered as a 20 mg intravenous bolus (over ˜3 minutes) with a continuous intravenous infusion of 60 mg/hour for 1 hour followed by a continuous intravenous infusion of 42 mg/hour for 23 hours. On days 2-5, the intravenous infusion of 42 mg/hour is continued until intravenous anesthesia is discontinued and the ganaxolone taper begins. Intravenous anesthesia is weaned beginning 4 hours after the start of the continuous infusion and intravenous anesthesia is discontinued about 48 hours to about 120 hours later (i.e., about day 3-5 of the regimen).

When intravenous anesthesia is discontinued (i.e., day 3-5 of the regimen) the continuous infusion of ganaxolone is tapered over a 2-4 day period so that the total number of days of infusion of ganaxolone does not exceed 7 days. To taper the ganaxolone continuous infusion the infusion rate is decreased every 4 hours in even increments to ensure discontinuation of the intravenous ganaxolone by the end of day 7. The continuous infusion will be discontinued when the rate reaches 10 mg/hr. As an illustrative example, if the taper is initiated after day 3, the dose is decreased by 1.68 mg/h every 4 hours for 96 hours at which time the continuous infusion is discontinued.

Subjects Weighing Less than 40 kg

On day 1 of the regimen, Ganaxolone is administered as a 0.5 mg/kg intravenous bolus (over ˜3 minutes) with a continuous intravenous infusion of 1.5 mg/kg/hour for 1 hour followed by a continuous intravenous infusion of 1.05 mg/kg/hour for 23 hours. On days 2-5, the intravenous infusion of 1.05 mg/kg/hour is continued until intravenous anesthesia is discontinued and the ganaxolone taper begins. Intravenous anesthesia is weaned beginning 4 hours after the start of the continuous infusion and intravenous anesthesia is discontinued about 48 hours to about 120 hours later (i.e., about day 3-5 of the regimen).

When intravenous anesthesia is discontinued (i.e., day 3-5 of the regimen) the continuous infusion of ganaxolone is tapered over a 2-4 day period so that the total number of days of infusion of ganaxolone does not exceed 7 days. To taper the ganaxolone continuous infusion the infusion rate is decreased every 4 hours in even increments to ensure discontinuation of the intravenous ganaxolone by the end of day 7. The continuous infusion will be discontinued when the rate reaches 10 mg/hr. As an illustrative example, if the taper is initiated after day 3, the dose is decreased by 0.042 mg/kg/h every 4 hours for 96 hours at which time the continuous infusion is discontinued.

Conversion from Intravenous to Oral Ganaxolone

Subjects weighing more than 28 kg may be converted to oral ganaxolone when intravenous ganaxolone is discontinued. It is recommended that patients start oral ganaxolone (via a NG tube if needed) at the beginning of the intravenous ganaxolone taper. The oral dose should start at 1,800 mg/day in three divided doses, and the dose is decreased as needed for any adverse reactions. Oral dosing is continued for 3-6 months followed by a 2-3 week taper. Suggested oral dosing and taper are shown in Table 1.

Subjects weighing 28 kg or less may be converted to oral ganaxolone when intravenous ganaxolone is discontinued. It is recommended that patients start oral ganaxolone (via a NG tube if needed) at the beginning of the intravenous ganaxolone taper. The oral dose should start at 63 mg/kg/day in three divided doses, and the dose is decreased as needed for any adverse reactions. Oral dosing is continued for 3 months followed by a 2-3 week taper. Suggested oral dosing and taper are shown in Table 2.

### Example 2: Case Report

A 34-year-old man with cerebral palsy and epilepsy (last known seizures in childhood), was admitted with acute hypoxic respiratory failure in setting of COVID-19 pneumonia for which he was treated with remdesivir and a course of dexamethasone. On hospital day 2, he had a witnessed tonic-clonic seizure without return to baseline followed by cEEG consistent with non-convulsive status epilepticus, for which he was urgently administered benzodiazepines, levetiracetam, and intravenous anesthetics (propofol and midazolam) in rapid succession. On hospital day 4, additional antiseizure medications (ASMs) were added (lacosamide, phenobarbital) for super refractory status epilepticus upon attempts to wean anesthetics. Between hospital day 5 and 21, he had continued titration of ASMs and intravenous anesthetics, as well as three days of methylprednisolone without lasting benefit. During this time, MRI brain with/without contrast and lumbar puncture were completed without evidence of underlying infectious or inflammatory etiology.

He was transferred to the neuro-intensive care unit on hospital day 22, following which additional interventions included ketamine infusion, further addition and up-titration of ASMs, ketogenic diet, plasma exchange and electroconvulsive therapy (FIG. 1). Despite these treatments, the patient's EEG had near continuous 2-4 Hz generalized periodic discharges on the ictal end of the ictal-interictal continuum without improvement in mental status. He rarely opened his eyes to noxious stimuli. He did not attend to examiners or family members and did not follow any commands.

On hospital day 52, intravenous ganaxolone (GNX) was administered as a bolus of 20 mg then infused at 60 mg/hr for the first hour, followed by 42 mg/hr for a total of 7 days of treatment, followed by enteral GNX as three times daily dosing. Predicted and observed plasma ganaxolone concentrations for the patient are shown in FIG. 2.

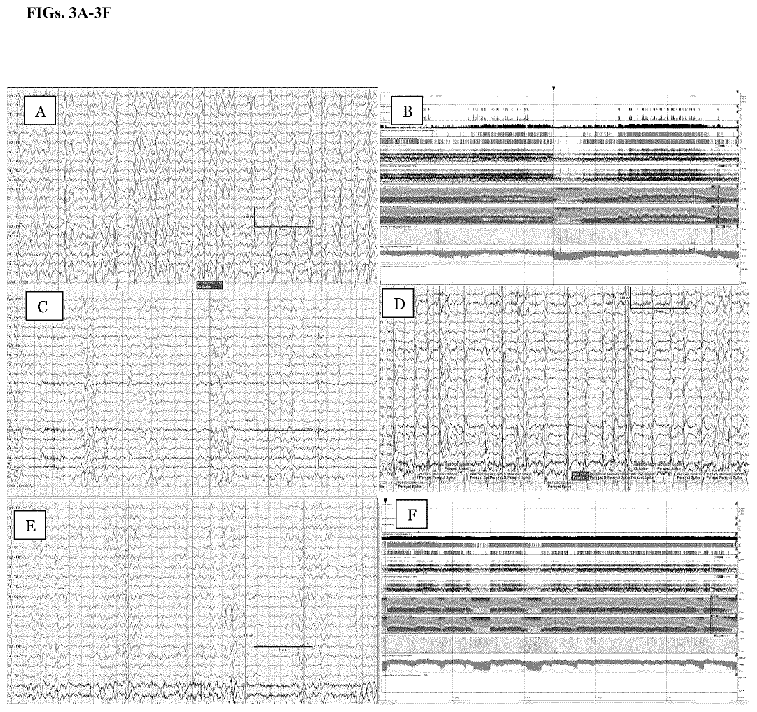

Prior to initiation of GNX, the EEG showed stimulus induced, fluctuating 2-4 Hz spiky, generalized periodic discharges (GPDs), at times meeting criteria for electrographic status epilepticus. During the time of GNX infusion, a marked improvement in the EEG pattern was recorded and in subsequent days, additional EEG data revealed an overall decrease in the frequency of generalized discharges though lower frequency GPDs remained, up to 1 Hz (FIGS. 3A-3F). Simultaneously, his mental status returned to his premorbid baseline (eye opening, attending, communicating with gestures and facial expressions) and we were able to wean ASMs, including discontinuation of phenobarbital.

The patient was discharged to a ventilator weaning facility on hospital day 88 (36 days after GNX was initiated). At follow up approximately one month after discharge, he remained fully dependent for activities of daily living with persistent need for tracheostomy and gastrostomy. He was awake, alert, and at times able to track and attend, remaining non-verbal without command following, and he regained a social smile with family, who reported him to be near his cognitive baseline. For seizure prophylaxis, GNX 600 mg TID was continued (plan to off titrate after a total of 6 weeks), as well as levetiracetam, lacosamide, clonazepam and perampanel, though these medications were overall able to be weaned during the post-acute period.

Despite late initiation of GNX, this patient showed electrographic and clinical improvement with return to his premorbid baseline mental status. Following administration of the initial ganaxolone bolus, peak serum ganaxolone concentration correlated with EEG improvement with overall less frequent generalized discharges, after which time there was a rebound worsening in discharge frequency correlating with a drop in GNX concentration, which lasted until there was further rise in ganaxolone to steady state with the continued infusion. The ganavolone treatment in this case came after nearly two months with ongoing epileptic activity which was refractory to maximal multimodal treatment, and led to an improvement in ictal activity on concurrent EEG recording. In this case, the patient had a good outcome.

1. A method for treating super refractory status epilepticus (SRSE), comprising administering to a subject in need thereof ganaxolone as an intravenous bolus plus continuous infusion, wherein the subject in need thereof is receiving intravenous anesthesia, and wherein
when the subject in need thereof weighs 40 kg or more the intravenous bolus comprises about 20 mg ganaxolone, and the continuous infusion comprises an infusion of about 60 mg ganaxolone/hr for about 1 hour and then about 42 mg ganaxolone/hr for about 23 hours; and
when the subject in need thereof weighs less than 40 kg the bolus comprises about 0.5 mg ganaxolone/kg, and the continuous infusion comprises an infusion of about 1.5 mg ganaxolone/kg/hr for about 1 hour and then about 1.05 mg ganaxolone/kg/hr for about 23 hours.

2. The method of claim 1, wherein the continuous infusion comprising about 42 mg ganaxolone/hr is continued from an additional about 24 to about 96 hours, or the continuous infusion comprising about 1.05 mg ganaxolone/kg/hr is continued from an additional about 24 to about 96 hours.

3. The method of claim 1, wherein the intravenous bolus is administered over about 3 minutes.

4. The method of claim 1, wherein the subject in need thereof is weaned off the intravenous anesthesia beginning about 4 hours after the start of the continuous infusion and intravenous anesthesia is discontinued about 48 hours to about 120 hours later.

5. The method of claim 4, wherein the continuous infusion is tapered and the taper starts about when intravenous anesthesia is discontinued.

6. The method of claim 5, wherein the continuous infusion of ganaxolone is tapered over a period of about 2-4 days and discontinued no later than about 7 days after the intravenous bolus was administered.

7. The method of claim 5, wherein the taper comprises decreasing the amount of ganaxolone that is infused every 4 hours in even increments to achieve discontinuation.

8. The method of claim 1, wherein the taper is complete and the continuous infusion is discontinued when the amount of ganaxolone infused is reduced to about 10 mg/hr.

9. The method of claim 1, wherein the taper begins on day 3, 4 or 5 after the intravenous bolus was administered and the taper is complete and the continuous infusion is discontinued on day 7 after the intravenous bolus was administered.

10. The method of claim 1, wherein following discontinuation of the continuous infusion oral ganaxolone is administered.

11. The method of claim 10, wherein oral ganaxolone is administered at a dose of 1,800 mg/day for subjects that weigh more than 28 kg; and at a dose of 63 mg/kg for subjects that weigh 28 kg or less.

12. The method of claim 10, wherein the oral ganaxolone is administered in 3 divided doses each day.

13. The method of claim 10, wherein the oral ganaxolone is administered for about 3-6 months.

14. The method of claim 13, wherein oral ganaxolone is administered for about 3-6 months to subjects that weigh more than 28 kg, and about 3 months to subjects that weigh 28 kg or less.

15. The method of claim 10, wherein oral ganaxolone is tapered over a period of 2-3 weeks and then discontinued.
